(19) United States
(12) Patent Application Publication
(10) Pub. No.: US 2023/0200631 A1
(43) Pub. Date: Jun. 29, 2023

(54) INSERTION ASSIST TUBE FOR ENDOSCOPE

(71) Applicant: FUJIFILM Corporation, Tokyo (JP)

(72) Inventors: Takumi DEJIMA, Kanagawa (JP); Tomohiro OHKI, Kanagawa (JP); Kenji NAGAMIYA, Kanagawa (JP); Shozo IYAMA, Kanagawa (JP)

(73) Assignee: FUJIFILM Corporation, Tokyo (JP)

(21) Appl. No.: 18/175,392

(22) Filed: Feb. 27, 2023

Related U.S. Application Data
(63) Continuation of application No. PCT/JP2021/031161, filed on Aug. 25, 2021.

(30) Foreign Application Priority Data
Aug. 31, 2020 (JP) 2020-145855

(51) Int. Cl. A61B 1/00; A61B 1/31

(52) U.S. Cl. CPC A61B 1/00154; A61B 1/31; A61B 1/0008

(57) ABSTRACT

An insertion assist tube for an endoscope includes an insertion assist tube body and a fluid suppression member. The insertion assist tube body has a pipe line through which an insertion part is inserted, and an opening portion. The fluid suppression member is provided in the opening portion to suppress passage of a fluid. The fluid suppression member has a first porous member on which a first slit is formed, and a second porous member on which a second slit intersecting the first slit is formed.

### CROSS-REFERENCE TO RELATED APPLICATIONS

This application is a Continuation of PCT International Application No. PCT/JP2021/031161 filed on 25 Aug. 2021, which claims priority under 35 U.S.C. § 119(a) to Japanese Patent Application No. 2020-145855 filed on 31 Aug. 2020. The above application is hereby expressly incorporated by reference, in its entirety, into the present application.

### BACKGROUND OF THE INVENTION

### 1. Field of the Invention

The present invention relates to an insertion assist tube for an endoscope that assists the insertion of an endoscope.

### 2. Description of the Related Art

In the related art, in the medical field, a procedure of inserting an insertion part of an endoscope into a lower gastrointestinal tract such as a large intestine to observe, diagnose, or treat an inner wall surface of the tract has been performed. The lower gastrointestinal tract is bent in a complicated manner, and it may be difficult to transmit a force to the insertion part simply by pushing the insertion part of the endoscope, and it may be difficult to insert the insertion part into the lower gastrointestinal tract.

Thus, in a case where the insertion part is inserted into the lower gastrointestinal tract, an insertion assist tube for an endoscope, which is referred to as an overtube or a sliding tube, may be used. The insertion assist tube for an endoscope has a pipe line through which the insertion part is inserted. By inserting the insertion assist tube for an endoscope into the subject through an anus, the insertion part of the endoscope can be easily inserted into the lower gastrointestinal tract through the pipe line (for example, JP3864344B (corresponding to US2005/124856A1)).

In a case where the insertion part of the endoscope is inserted through the pipe line of the insertion assist tube for an endoscope, the inside of the pipe line communicates with the lower gastrointestinal tract of a patient. Therefore, body fluids such as excrement in the intestine and blood may flow to the inside of the pipe line. The insertion assist tube for an endoscope described in JP3864344B has a liquid accumulation portion communicating with the pipe line, and a fluid leakage preventing unit such as a sponge formed in a donut shape is housed inside the liquid accumulation portion. Accordingly, the fluid leakage preventing unit and the insertion part are brought into close contact with each other to prevent leakage of a body fluid.

### SUMMARY OF THE INVENTION

In the medical field, it is desired to take further preventive measures against infectious diseases. In particular, it is important to take measures to prevent infection caused by contact of the body fluid of an infectious disease patient with mucous membranes of eyes, a mouth, or the like of a doctor or a helper, and infection caused by inhalation of droplets released from the infectious disease patient.

However, in the insertion assist tube for an endoscope described in JP3864344B, in a case where the insertion part is inserted into the pipe line, leakage of the body fluid can be prevented by the close contact of the fluid leakage preventing unit with the insertion part. However, in a case where the insertion part is removed from the insertion assist tube for an endoscope, the opening portion of the fluid leakage preventing unit is exposed. Thus, there is a possibility that the body fluid and the droplets released from the body of the patient may come into contact with the mucous membrane of a person around a patient, such as a doctor or a helper, through the opening portion of the exposed fluid leakage preventing unit.

An object of the present invention is to provide an insertion assist tube for an endoscope that can prevent leakage of a body fluid and droplets released from a subject in both a case where an insertion part of the endoscope is inserted through the anus of the subject and a case where the insertion part is removed.

The insertion assist tube for an endoscope according to the embodiment of the present invention comprises an insertion assist tube body and a fluid suppression member provided in an opening portion to suppress passage of a fluid in a pipe line, and the fluid suppression member is a first porous member and a second porous member. The insertion assist tube body is an insertion assist tube body that is inserted into a subject in a case where an insertion part of an endoscope is transanally inserted into the subject, has a pipe line through which the insertion part is inserted, and has an opening portion provided at an end of the pipe line on a side located outside a body of the subject in a case where the insertion assist tube body is inserted into the subject. The fluid suppression member is provided at the opening portion to suppress passage of a fluid in the pipe line. The first porous member has a first slit parallel to an insertion direction of the insertion part formed therein. The second porous member is formed with a second slit that is parallel to the insertion direction and that intersects the first slit.

It is preferable that three first slits are formed in the first porous member, and three second slits are formed in the second porous member.

It is preferable that the first porous member and the second porous member are fixed to the opening portion in a state in which the second slit is disposed at a position rotated by 180° around a central axis parallel to the insertion direction with respect to the first slit. It is preferable that the first porous member and the second porous member are integrally provided in a state where the first porous member and the second porous member are laminated.

It is preferable for a connection tube to be provided that is at a position of the insertion assist tube body closer to the subject than the fluid suppression member is, is disposed at a point located outside the body of the subject, communicates with the pipe line of the insertion assist tube, and is used for connection with an external device.

It is preferable that at least one of the connection tubes is a suction tube that is connected to a suction device serving as the external device and that suctions a liquid in the pipe line.

An insertion assist tube for an endoscope according to an aspect of the present invention comprises an insertion assist tube body, a fluid suppression member, and a connection tube, the fluid suppression member includes a duckbill valve and a thin plate member, the duckbill valve and the thin plate member are fixed to the opening portion in a state in which the duckbill valve and the thin plate member are laminated, and at least one of the connection tubes is a suction tube that is connected to a suction device serving as the external device and that suctions a liquid in the pipe line. The duckbill valve maintains airtightness of the pipe line in a state in which the insertion part is not inserted. The thin plate member has a through-hole with an inner diameter that matches an outer diameter of the insertion part and maintains the airtightness of the pipe line in a state in which the insertion part is inserted.

It is preferable for a plurality of the connection tubes to be provided, and it is preferable that at least one of the connection tubes is a liquid feeding tube that is connected to a liquid feeding device serving as the external device and that supplies a liquid into the pipe line.

It is preferable that the fluid suppression member has a larger dimension than an outer diameter of the insertion assist tube body. It is preferable that the insertion assist tube body has a tubular portion to be inserted into an anus of the subject and a flange portion consecutively installed at the tubular portion, and the flange portion protrudes from an outer peripheral surface of the tubular portion.

According to the present invention, in both a case where the insertion part of the endoscope is inserted through the anus of the subject and a case where the insertion part is removed, the leakage of a body fluid and droplets can be prevented in a case where the insertion part of the endoscope is inserted through the anus of the subject.

### BRIEF DESCRIPTION OF THE DRAWINGS

FIG. 1 is a schematic view showing an endoscopy using an endoscope for observing a lower gastrointestinal tract and an insertion assist tube for an endoscope.

FIG. 2 is a perspective view of the insertion assist tube for an endoscope.

FIG. 3 is an exploded perspective view of the insertion assist tube for an endoscope.

FIG. 4 is a perspective view showing a configuration of a fluid suppression member.

FIG. 5 is an explanatory diagram illustrating a state in which an insertion part is inserted into the fluid suppression member.

FIG. 6 is a cross-sectional view of major parts cut along a center line in the insertion assist tube for an endoscope.

FIG. 7 is a cross-sectional view of major parts cut along a line VII-VII of FIG. 6.

FIG. 8 is an explanatory diagram illustrating an operation in a case where an endoscopy is performed on a subject into which the insertion assist tube for an endoscope is inserted.

FIG. 9 is a perspective view of the insertion assist tube for an endoscope according to a first modification example.

FIG. 10 is a perspective view showing a configuration of a fluid suppression member in the first modification example.

FIG. 11 is a perspective view of an insertion assist tube for an endoscope of a second embodiment.

FIG. 12 is an exploded perspective view showing a configuration of the insertion assist tube for an endoscope of the second embodiment.

FIGS. 13A and 13B are perspective views showing a configuration of a fluid suppression member in a third embodiment.

FIG. 14 is an exploded perspective view showing a configuration of the insertion assist tube for an endoscope of the third embodiment.

FIG. 15 is a cross-sectional view of major parts cut along a center line in the insertion assist tube for an endoscope of the third embodiment.

FIG. 16 is a cross-sectional view of major parts cut along a center line in an insertion assist tube for an endoscope of a second modification example.

FIG. 17 is a cross-sectional view of major parts cut along a center line in an insertion assist tube for an endoscope of a third modification example.

FIG. 18 is a cross-sectional view of major parts cut along a center line in an insertion assist tube for an endoscope of a fourth modification example.

FIG. 19 is a cross-sectional view of major parts cut in a direction perpendicular to an insertion direction in an insertion assist tube for an endoscope of a fifth modification example.

### DESCRIPTION OF THE PREFERRED EMBODIMENTS

### First Embodiment

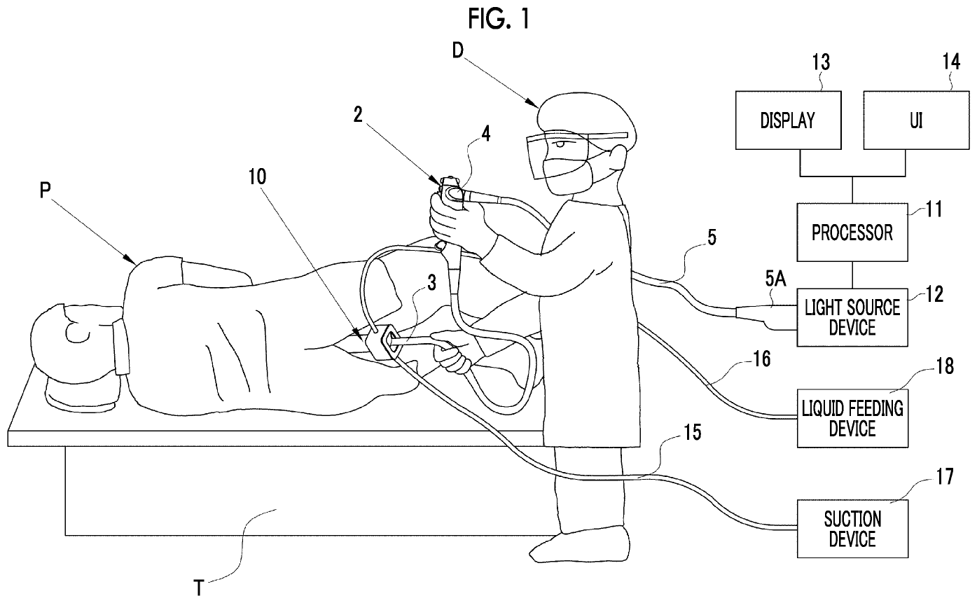

As shown in FIG. 1, an insertion assist tube 10 for an endoscope (hereinafter, referred to as an insertion assist tube 10) according to embodiments of the present invention is used in an endoscopy for observing a lower gastrointestinal tract such as a large intestine. The endoscope 2 includes an insertion part 3, an operating part 4, and a universal cord 5. The insertion part 3 is inserted transanally into the lower gastrointestinal tract of a subject P who is a test object. The operating part 4 is consecutively installed in a proximal end part of the insertion part 3. The universal cord 5 is connected to the operating part 4.

In addition, FIG. 1 shows a state in which a doctor D, who is a user, grips the operating part 4 with one hand and the insertion part 3 with the other hand, and inserts the insertion part 3 into a body of the subject P through the insertion assist tube 10. The subject P wears, for example, an examination garment having an opening portion or a notch formed on a rear surface. Additionally, the subject P is undergoing the examination in a lateral decubitus posture (a state of lying sideways) on an examination table T.

The universal cord 5 is connected to an external device such as a processor device or a light source device via a connector 5A. A processor device 11 is electrically connected to a display 13 and to a user interface (UI) 14. The UI 14 includes a keyboard, a mouse, a touch pad, a microphone, and the like, and receives an input operation of the doctor D who is a user.

Although not shown, a distal end surface of the insertion part 3 is provided with an observation window and an illumination window. An image sensor (not shown) or the like is disposed behind the observation window, and an optical fiber cable (not shown) is disposed behind the illumination window. A signal line of the image sensor and the optical fiber cable are connected to the processor device 11 and to a light source device 12, respectively, through the insertion part 3, the operating part 4, and the universal cord 5. The processor device 11 performs image processing or the like on an endoscopic image captured by the image sensor and displays the image-processed image on the display 13.

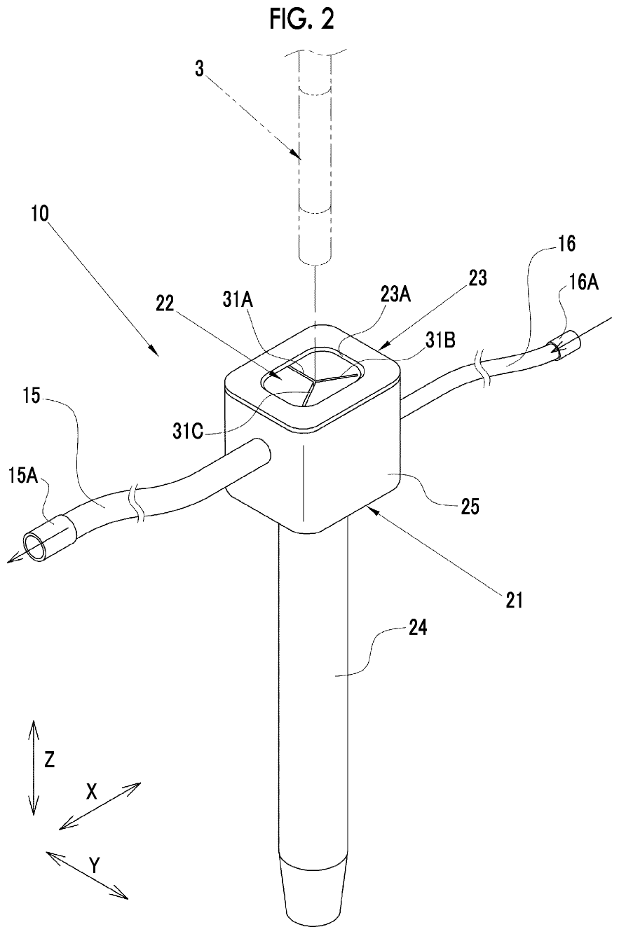

As shown in FIG. 2, the insertion assist tube 10 comprises an insertion assist tube body 21, a fluid suppression member 22, a retaining member 23, a suction tube 15, and a liquid feeding tube 16.

In addition, the suction tube 15 and the liquid feeding tube 16 correspond to connection tubes within the claims.

The suction tube 15 is connected to a suction device 17 via a connection connector 15A. The suction device 17 is operated by the operation of the doctor D or a helper to generate a negative pressure. By operating the suction device 17, a liquid inside the insertion assist tube 10 is suctioned through the suction tube 15. In addition, the suction device 17 may be a drain that performs suction via the manual operation of the doctor D or a helper or may be a suction pump that automatically generates a negative pressure.

The liquid feeding tube 16 is connected to a liquid feeding device 18 via a connection connector 16A. By operating the liquid feeding device 18, a cleaning liquid is fed to the inside of the insertion assist tube 10 through the liquid feeding tube 16. As the liquid feeding device 18, for example, a liquid feeding device, such as a liquid feeding pump, similar to one for feeding the liquid to the endoscope 2 is used.

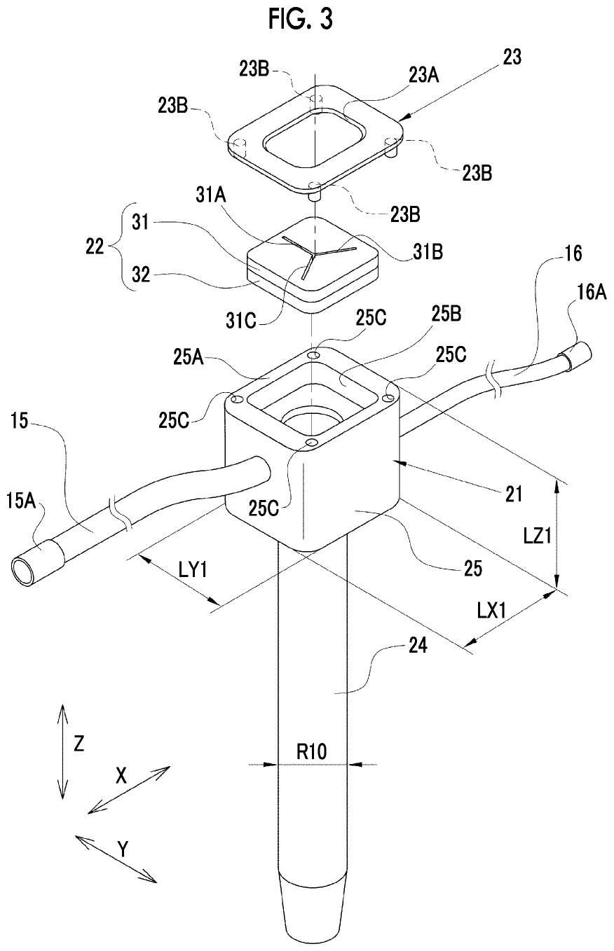

As shown in FIG. 3, in the insertion assist tube body 21, a tubular portion 24 and a flange portion 25 are integrally provided. The insertion assist tube body 21 is formed of, for example, a soft material such as a transparent or translucent soft resin. The tubular portion 24 is a portion that is inserted into an anus of the subject P in a case where the insertion part 3 of the endoscope 2 is transanally inserted into the subject P. The tubular portion 24 is formed to have a circular cross section so that the tubular portion 24 can be easily inserted into the anus. In addition, the cross section of the tubular portion 24 is not limited to these examples and may have an elliptical shape.

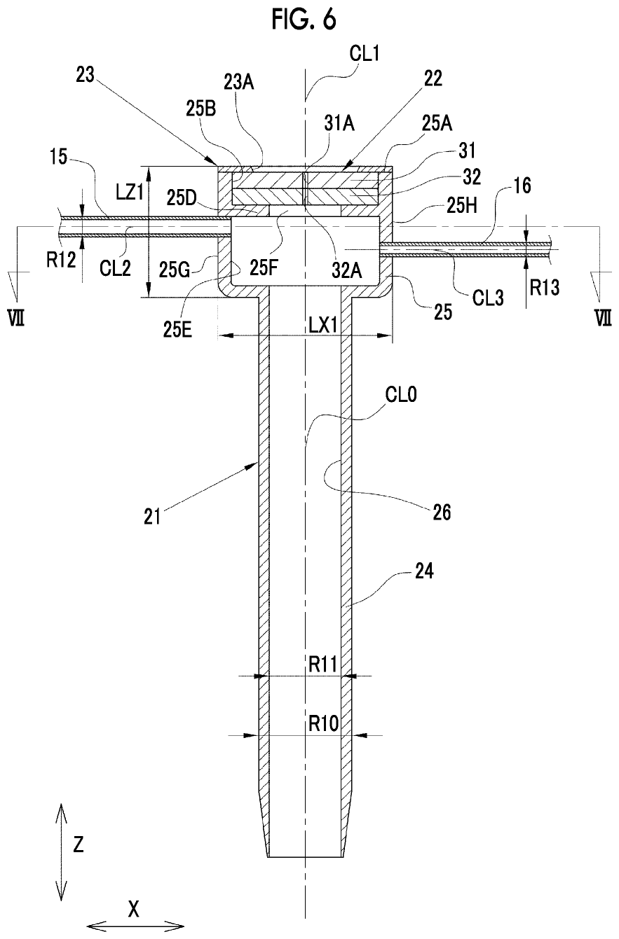

The flange portion 25 is consecutively installed at a proximal end of the tubular portion 24 and is formed in a box shape. Hereinafter, in a case where the tubular portion 24 is inserted into the anus, an end located outside the body of the subject P is referred to as a proximal end in an insertion direction Z, and an end located inside the body of the subject P is referred to as a distal end in the insertion direction Z. A pipe line 26 (refer to FIG. 6) through which the insertion part 3 is inserted is provided inside the tubular portion 24 and the flange portion 25. The pipe line 26 is a through-hole that is disposed in the insertion direction Z and has a circular or elliptical cross section.

The flange portion 25 has a box shape in which a proximal end 25A is open. The proximal end 25A is formed in a quadrangular shape, and an opening portion 25B is formed. The opening portion 25B is a quadrangular opening portion formed in conformity with the outer shape of first and second porous members 31 and 32 which will be described below. Hereinafter, a direction parallel to one side of a quadrangle constituting the opening portion 25B will be referred to as a left-right direction X, and a direction parallel to another side forming the opening portion 25B and perpendicular to the left-right direction X will be described as a front-back direction Y. Additionally, the insertion direction Z is a direction perpendicular to the left-right direction X and to the front-back direction Y, and is parallel to a center line CL0 (refer to FIG. 6) of the tubular portion 24. A fitting hole 25C into which a fitting pin 23B to be described below is fitted is formed around the opening portion 25B.

The opening portion 25B is provided with the fluid suppression member 22. In a case where the tubular portion 24 is inserted into the anus, the fluid suppression member 22 suppresses the passage of a fluid in the pipe line 26 leading to the inside of the body of the subject P. Specifically, the fluid suppression member 22 allows a gas such as air to pass through and blocks a liquid such as a body fluid and droplets. The fluid suppression member 22 includes a first porous member 31 and a second porous member 32, and three slits are disposed in a Y shape.

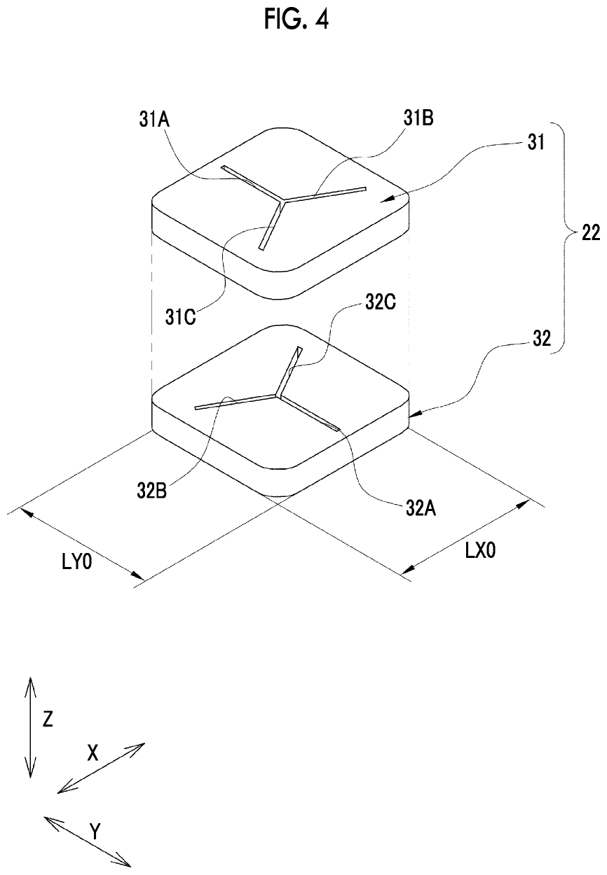

As shown in FIG. 4, the first porous member 31 has three first slits 31A to 31C. The first porous member 31 is formed by forming a flexible porous material into a quadrangular plate shape. The first slits 31A to 31C are disposed in a Y shape. More specifically, the first slits 31A to 31C are disposed at equal angle intervals of 120° and are connected to each other at the center of the first porous member 31. One first slit 31A of the first slits 31A to 31C is perpendicular to one side of the first porous member 31, that is, perpendicular to the left-right direction X, and is disposed parallel to the front-back direction Y.

The second porous member 32 is formed by forming a flexible porous material into a quadrangular plate shape. The second porous member 32 has three second slits 32A to 32C. The second slits 32A to 32C are formed in a direction parallel to the insertion direction Z and intersecting the first slits 31A to 31C. Specifically, the disposition of the second porous member 32 is the same as that of the first porous member 31 rotated by 180° around the central axis parallel to the insertion direction Z. Similarly to the first slits 31A to 31C, the second slits 32A to 32C are disposed at equal angle intervals of 120° and are connected at the center of the second porous member 32. Accordingly, the second slits 32A to 32C are disposed at positions rotated by 180° around the central axis parallel to the insertion direction Z with respect to the first slits 31A to 31C. One second slit 32A of the second slits 32A to 32C is perpendicular to one side of the second porous member 32, that is, perpendicular to the left-right direction X, and is disposed parallel to the front-back direction Y.

The porous material that forms the first and second porous members 31 and 32 is a porous material having a pore diameter and a structure that allows a gas such as air to pass therethrough and that blocks liquids, such as the body fluid, and the droplets. For example, a synthetic sponge obtained by foam-molding resin or a natural sponge such as a sea sponge is used. The first porous member 31 and the second porous member 32 may be separately formed, and may be bonded and joined in a state in which the porous members are rotated by 180° around the central axis parallel to the insertion direction Z. Cost reduction can be achieved by using two of the same porous members in this way.

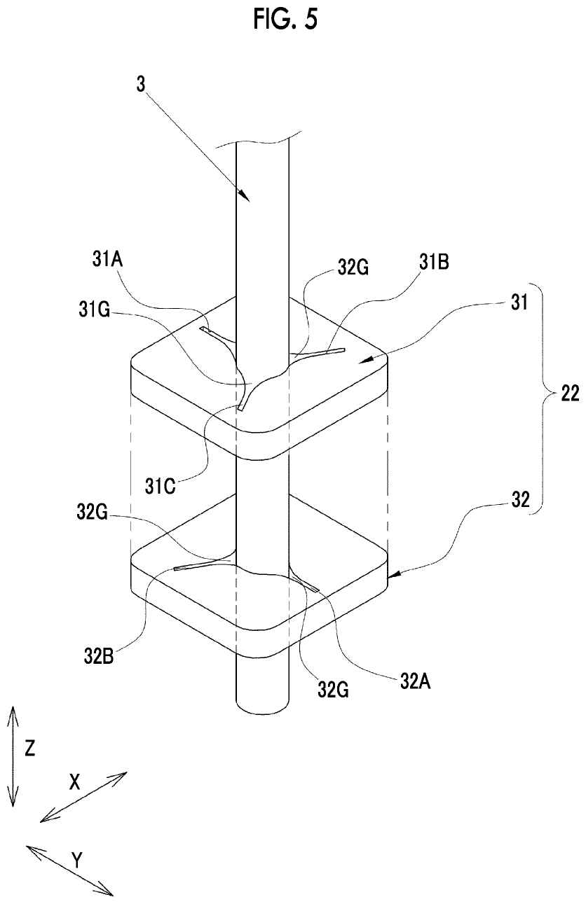

As shown in FIG. 5, in a case where the insertion part 3 is inserted into the fluid suppression member 22, the insertion part 3 moves in the insertion direction Z while an outer peripheral surface of the insertion part 3 comes into close contact with the first slits 31A to 31C and the second slits 32A to 32C. In this case, gaps 31G and 32G are formed in some of the first slits 31A to 31C and the second slits 32A to 32C. However, as described above, since the second slits 32A to 32C are disposed by being rotated 180° around the central axis parallel to the insertion direction Z with respect to the first slits 31A to 31C, the positions of the gaps 31G and 32G do not overlap each other. Thus, it is possible to prevent the body fluid and the droplets from leaking from the gaps 31G and 32G.

In this case, by providing the first porous member 31 and the second porous member 32 with the three first slits 31A to 31C and the three second slits 32A to 32C, respectively, instead of one slit, it is possible to insert the insertion part 3 with a resistance smaller than that in a case where each porous member is provided with one slit. Moreover, it is possible to insert a smaller-diameter insertion part 3 having a diameter of about 3 mm to a large-diameter insertion part 3 having a diameter of about 16 mm with a small resistance without any gap. Accordingly, it is possible to form the first porous member 31 and the second porous member 32 from a porous material having a high density (having fewer voids), and the leakage of the body fluid and the droplets can be further suppressed.

Additionally, it is preferable that the outer shape of the fluid suppression member 22, that is, lengths LX0 and LY0 (refer to FIG. 4) of the respective sides of the first and second porous members 31 and 32, has a larger dimension than an outer diameter R10 of the tubular portion 24. Accordingly, the fluid suppression member 22 can sufficiently suppress the passage of the fluid.

As shown in FIG. 6, the flange portion 25 is disposed at a position where the center line CL0 of the tubular portion 24 and the center line CL1 of the flange portion 25 coincide with each other in the left-right direction X and in the front-back direction Y. The flange portion 25 has lengths LX1 and LY1 (refer to FIG. 3) in the left-right direction X and in the front-back direction Y larger than the outer diameter R10 of the tubular portion 24 and protrudes from an outer peripheral surface of the tubular portion 24. Accordingly, in a case where the tubular portion 24 is inserted into the anus, the flange portion 25 abuts against a body surface of the subject P. Thus, the insertion assist tube 10 is stopped at a position where the flange portion 25 abuts against the body surface of the subject P. The flange portion 25 is located outside the body of the subject P in a case where the tubular portion 24 is inserted into the anus.

In addition, an inner diameter R11 of the pipe line 26 is formed in accordance with the outer diameter of the insertion part 3, and is preferably 15 mm, for example. Additionally, it is preferable that the dimensions of the flange portion 25 are 30 mm to 50 mm in the lengths LX1 and LY1 in the left-right direction X and in the front-back direction Y, and it is preferable that a length LZ1 in the insertion direction is 20 mm to 50 mm.

In addition to the opening portion 25B, a partition plate 25D and a storage portion 25E are formed inside the flange portion 25. The storage portion 25E is at a position closer to a distal end side in the insertion direction Z than the fluid suppression member 22 is, that is, closer to the inside of the body of the subject P than the opening portion 25B is in a case where the tubular portion 24 is inserted into the anus. The storage portion 25E constitutes a part of the pipe line 26 through which the insertion part 3 is inserted, and the dimensions thereof in the left-right direction X and in the front-back direction Y are larger than the inner diameter R11 of the pipe line 26. For this reason, in a case where the tubular portion 24 is inserted into the anus, body fluids such excrement in the intestine and blood released from the body of the subject P flow into the tubular portion. Additionally, by operating the liquid feeding device 18, the storage portion 25E is supplied with, for example, a cleaning liquid such as water via the liquid feeding tube 16.

The partition plate 25D partitions between the opening portion 25B and the storage portion 25E. In a case where the opening portion 25B and the outer peripheral surfaces of the first and second porous members 31 and 32 are fitted to each other, an end surface of the second porous member 32 abuts against the partition plate 25D. A through-hole 25F through which the insertion part 3 is inserted is formed in the partition plate 25D. The through-hole 25F communicates with the inside of the opening portion 25B and the storage portion 25E.

The retaining member 23 is formed in a quadrangular frame shape. The retaining member 23 is formed in conformity with the outer shape of the flange portion 25 and is anchored to the proximal end 25A of the flange portion 25. The retaining member 23 has a through-hole 23A and four fitting pins 23B (refer to FIG. 3). The through-hole 23A exposes the first slits 31A to 31C of the first porous member 31.

In a case where the fluid suppression member 22 is attached to the insertion assist tube body 21, the opening portion 25B of the flange portion 25 and the outer peripheral surfaces of the first and second porous members 31 and 32 are fitted to each other, and the first and second porous members 31 and 32 are sandwiched between the retaining member 23 and the insertion assist tube body 21. Accordingly, the first porous member 31 and the second porous member 32 are provided integrally with the insertion assist tube body 21 and the retaining member 23 in a laminated state, and the fluid suppression member 22 is fixed to the insertion assist tube body 21. In this case, the insertion assist tube body 21 and the retaining member 23 are anchored to each other by fitting the fitting pin 23B formed in the retaining member 23 into the fitting hole 25C formed in the flange portion 25.

In addition, the method of anchoring the insertion assist tube body 21 and the retaining member 23 to each other is not limited to the above method. For example, the insertion assist tube body 21 and the retaining member 23 may be anchored to each other by bonding or pressure bonding. Additionally, a fitting hole may be formed in the retaining member 23, and a fitting pin may be formed in the flange portion 25 to fit the two to each other.

In addition, in the present embodiment, in a state in which the first porous member 31 and the second porous member 32 constituting the fluid suppression member 22 are laminated, the fluid suppression member 22 is attached to the flange portion 25 provided in the insertion assist tube body 21. However, the present invention is not limited thereto. For example, the integrally provided fluid suppression member 22 may be directly anchored to the proximal end of the tubular portion 24 without providing the flange portion 25 in the insertion assist tube body 21.

The suction tube 15 is connected to one side surface 25G of the flange portion 25, and the liquid feeding tube 16 is connected to a side surface 25H different from the side surface 25G. The suction tube 15 and the liquid feeding tube 16 are provided at a point where the tubes communicate with the storage portion 25E. That is, the suction tube 15 and the liquid feeding tube 16 are at positions closer to the inside of the body of the subject P than the fluid suppression member 22 is in a case where the tubular portion 24 is inserted into the anus. The suction tube 15 and the liquid feeding tube 16 extend in a direction parallel to the left-right direction X with respect to the flange portion 25. An inner diameter R12 of the suction tube 15 needs to have a size such that the intestine is not clogged with excrement or the like, and is preferably 4 mm, for example. An inner diameter R13 of the liquid feeding tube 16 may be any size as long as the cleaning liquid can be supplied, and is preferably 3 mm, for example.

The suction tube 15 and the liquid feeding tube 16 have different connection positions with respect to the flange portion 25 in the insertion direction Z. Specifically, a center line CL3 of the liquid feeding tube 16 is at a position closer to the distal end side than the center line CL2 (refer to FIG. 6) of the suction tube 15 is in the insertion direction Z, and closer to the inside of the body of the subject P in a case where the tubular portion 24 is inserted into the anus. Moreover, the suction tube 15 is disposed at a position as close to the fluid suppression member 22 as possible, that is, at a position in a case where the suction tube 15 communicates with the storage portion 25E and is in contact with the partition plate 25D.

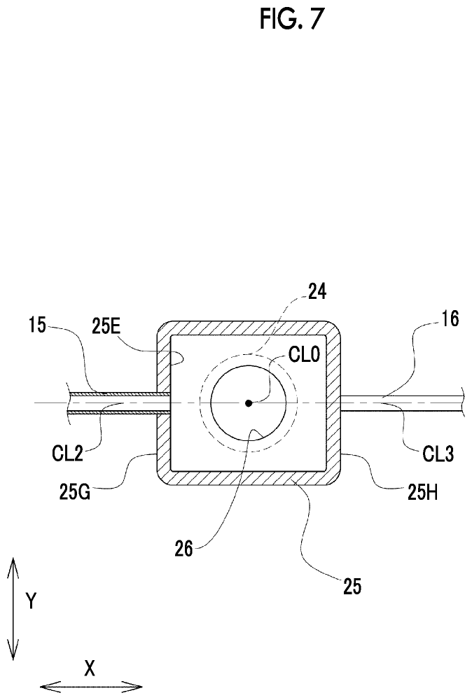

As shown in FIG. 7, the suction tube 15 and the liquid feeding tube 16 have different connection positions in the left-right direction X and the same connection positions in the front-back direction Y with respect to the flange portion 25. That is, the center line CL3 of the liquid feeding tube 16 is disposed at a position rotated by 180° around the center line CL0 of the tubular portion 24 with respect to the center line CL2 of the suction tube 15.

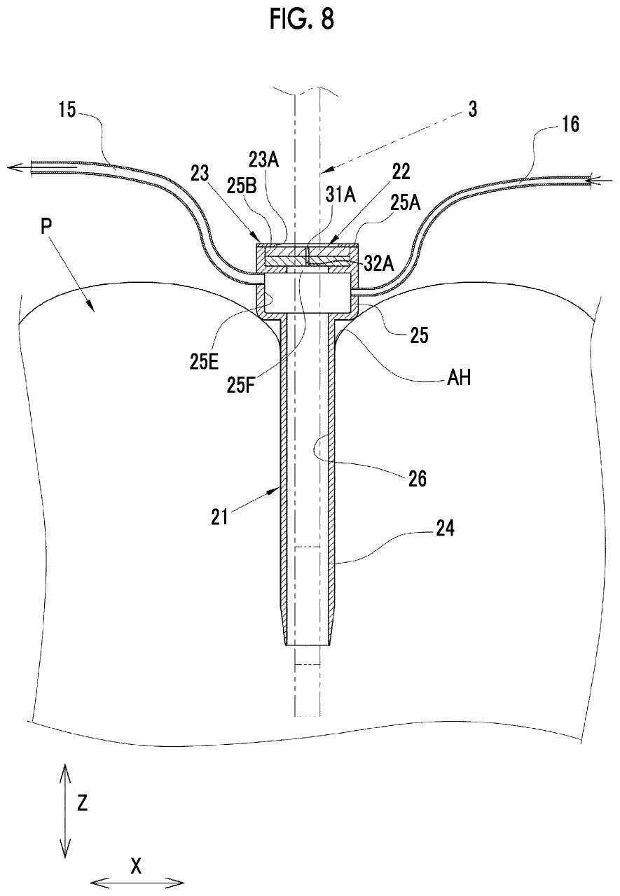

An operation in a case where the doctor D, who is a user, performs an endoscopy using the insertion assist tube 10 will be described. As shown in FIG. 8, first, as a preparation before the insertion part 3 is inserted into the subject P, the doctor D inserts the tubular portion 24 of the insertion assist tube 10 into an anus AH of the subject P. In this case, the subject P is in a lateral decubitus posture in which his/her left side faces downward on the examination table T (the state shown in FIG. 1). As described above, since the insertion assist tube 10 is provided with the flange portion 25, the flange portion 25 stops at a position where the flange portion 25 abuts against the body surface of the subject P. That is, it is possible to prevent the entire insertion assist tube 10 from entering the inside of the body of the subject P. Additionally, the doctor D connects the suction tube 15 to the suction device 17 and the liquid feeding tube 16 to the liquid feeding device 18.

Next, the doctor D inserts the insertion part 3 of the endoscope 2 from the anus AH into the body of the subject P, for example, the lower gastrointestinal tract such as the large intestine, through the insertion assist tube 10. The insertion part 3 is guided from the first slits 31A to 31C and the second slits 32A to 32C of the first and second porous members 31 and 32 to the pipe line 26 through the through-hole 25F and the storage portion 25E. The doctor D can easily insert the insertion part 3 into the lower gastrointestinal tract through the pipe line 26, that is, in the insertion direction Z.

In a case where an examination is performed on the lower gastrointestinal tract such as the large intestine with the endoscope 2, the body fluids, such as the excrement in the intestine and the blood, and the droplets are released together with air and cleaning liquid fed through the liquid feeding tube 16 and the endoscope 2. However, as described above, in the insertion assist tube 10, the insertion part 3 is inserted into the body of the subject P through the fluid suppression member 22. Accordingly, the fluid suppression member 22 suppresses the passage of the fluids. Thus, it is possible to prevent the body fluid and the droplets from leaking from the insertion assist tube 10.

Meanwhile, even in a case where the insertion part 3 is removed from the insertion assist tube 10, in the fluid suppression member 22, the second slits 32A to 32C are formed in the directions intersecting the first slits 31A to 31C. Thus, it is possible to prevent the body fluid and the droplets from leaking from the second slits 32A to 32C and the first slits 31A to 31C. Additionally, in the case of the fluid suppression member 22, the three first slits 31A to 31C and the three second slits 32A to 32C are guided to the centers of the first porous member 31 and the second porous member 32. Therefore, in a case where the insertion part 3 is inserted, it is not necessary to aim at the center, and the insertion can be easily performed.

Additionally, in a case where dirt resulting from the body fluid or the like is visually recognized during the endoscopy, the doctor D operates the liquid feeding device 18 to start feeding the cleaning liquid to the storage portion 25E. By feeding the cleaning liquid into the storage portion 25E, the insertion part 3 can be inserted while cleaning the inside of the pipe line 26. In addition, in this case, the suction device 17 may be operated to suction the body fluid or the like stored inside the storage portion 25E. It is preferable that the liquid feeding performed by the liquid feeding device 18 and the suction performed by the suction device 17 are not continuously performed, but are performed at a time when the doctor D or the helper recognizes the dirt resulting from the body fluid or the like.

Additionally, after the observation of the lower gastrointestinal tract by the endoscope 2 is completed, the doctor D operates the suction device 17 to suction the body fluid or the like stored inside the storage portion 25E when the insertion part 3 is removed from the insertion assist tube 10 or before the insertion part 3 is removed from the insertion assist tube 10. As described above, since the suction tube 15 is disposed at a position as close to the fluid suppression member 22 as possible, the body fluid and the droplets are suctioned from the suction tube 15 before reaching the fluid suppression member 22. Accordingly, the leakage of the body fluid and the droplets from the fluid suppression member 22 can be further prevented.

### First Modification Example

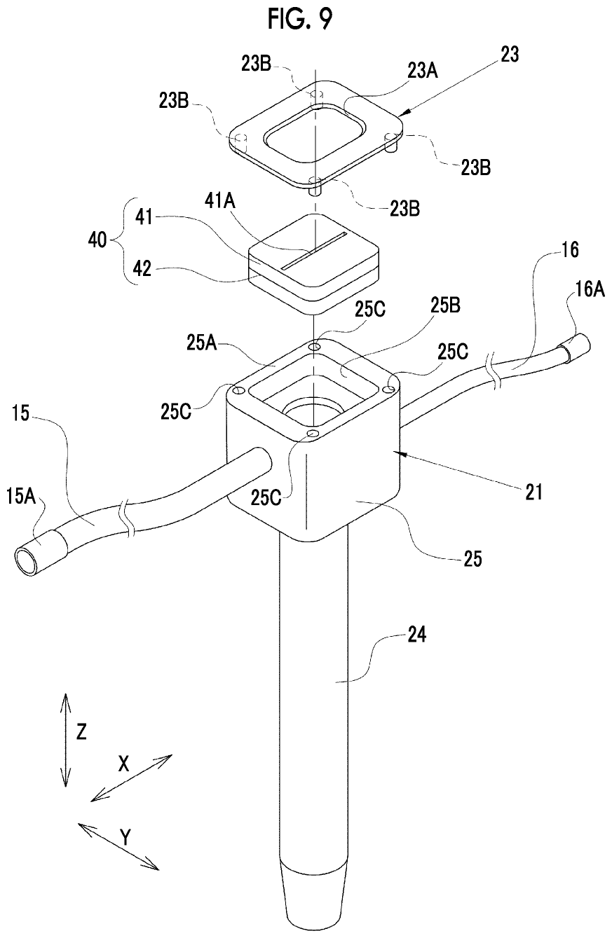

In the above first embodiment, an example is given in which the fluid suppression member include first and second porous members 31 and 32 in which three slits disposed in a Y shape are formed. However, the present invention is not limited to this, and in a modification example shown in FIG. 9, a fluid suppression member 40 includes first and second porous members 41 and 42 in which slits are formed one by one.

The first porous member 41 is formed by forming a flexible porous material into a quadrangular plate shape. The first porous member 41 has a first slit 41A. The first porous member 41 fits into the opening portion 25B of the flange portion 25. The first slit 41A is formed parallel to the left-right direction X and parallel to the insertion direction Z of the insertion part 3.

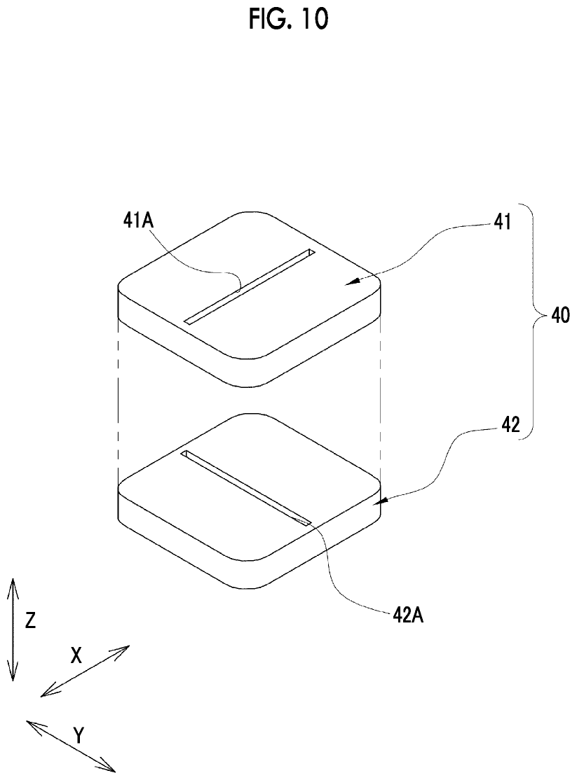

As shown in FIG. 10, the second porous member 42 is formed by forming a flexible porous material into a quadrangular plate shape. The second porous member 42 has a second slit 42A. The second porous member 42 fits into the opening portion 25B of the flange portion 25. The second slit 42A is formed in a direction parallel to the insertion direction Z and intersecting the first slit 41A. More specifically, the second slit 42A is formed parallel to the insertion direction Z and parallel to the front-back direction Y. In this way, since the first slit 41A and the second slit 42A intersect each other, the positions of the gaps with respect to the insertion part 3 do not overlap each other. Additionally, it is preferable that the length of each side of the first and second porous members 41 and 42 has a larger dimension than the outer diameter R10 of the tubular portion 24.

In addition, in the present modification example, the disposition of the second porous member 42 may be the same as that of the first porous member 41 rotated by 90° around the central axis parallel to the insertion direction Z. In this case, it is preferable that the outer shapes of the first and second porous members 41 and 42 are both square.

The porous materials that form the first and second porous members 41 and 42 are the same as the porous materials that form the first and second porous members 31 and 32 of the above first embodiment. It is preferable that the first porous member 41 and the second porous member 42 are separately formed and joined by bonding or the like in a state in which the second slit 42A is disposed in a direction intersecting the first slit 41A.

In a case where the fluid suppression member 40 is attached to the insertion assist tube body 21, similarly to the above first embodiment, the opening portion 25B of the flange portion 25 and the outer peripheral surfaces of the first and second porous members 41 and 42 are fitted to each other, and the first and second porous members 41 and 42 are sandwiched between the retaining member 23 and the insertion assist tube body 21. Accordingly, the fluid suppression member 40 is fixed to the insertion assist tube body 21 in a state in which the first porous member 41 and the second porous member 42 are laminated.

By using the insertion assist tube 10 provided with the fluid suppression member 40 for endoscopy, in a case where the insertion part 3 is inserted through the pipe line 26, similarly to the above first embodiment, the fluid suppression member 40 can suppress the passage of the fluid, and the body fluid and the droplets can be prevented from leaking. Additionally, even in a case where the insertion part 3 is removed from the insertion assist tube 10, the fluid suppression member 40 is formed in a direction in which the second slit 42A intersects the first slit 41A. Thus, the body fluid and the droplets can be prevented from leaking.

### Second Embodiment

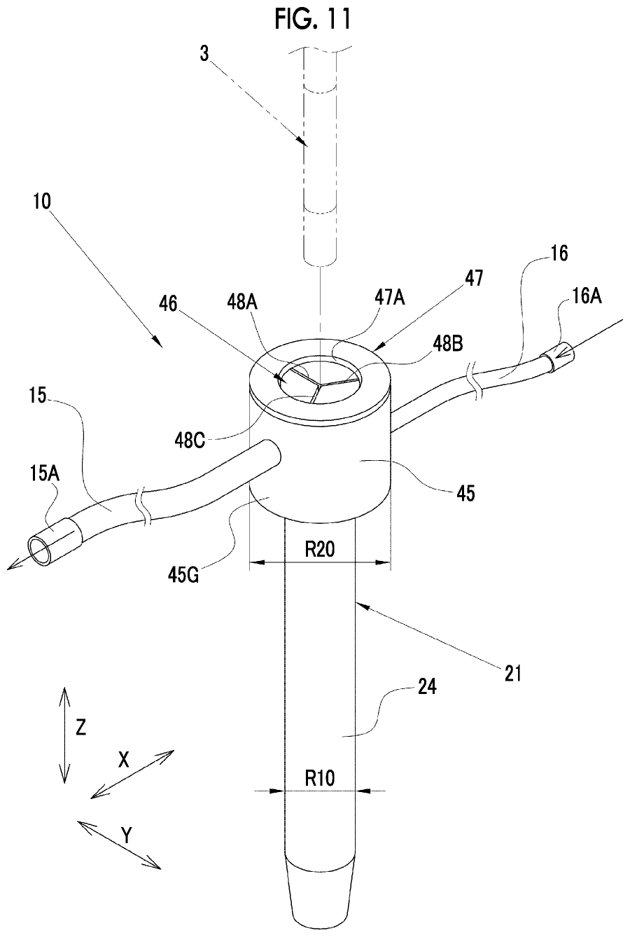

Additionally, in the above first embodiment, the flange portion 25 constituting the insertion assist tube body 21 is formed in a box shape. However, the present invention is not limited to this, and the flange portion forming the insertion assist tube body 21 may be formed in a cylindrical shape. As shown in FIG. 11, in the present embodiment, a fluid suppression member 46 provided in a flange portion 45 has a disk shape, and a retaining member 47 has an annular shape. Similarly to the flange portion 25 in the above first embodiment, the flange portion 45 is consecutively installed at the tubular portion 24 and is disposed coaxially with the tubular portion 24.

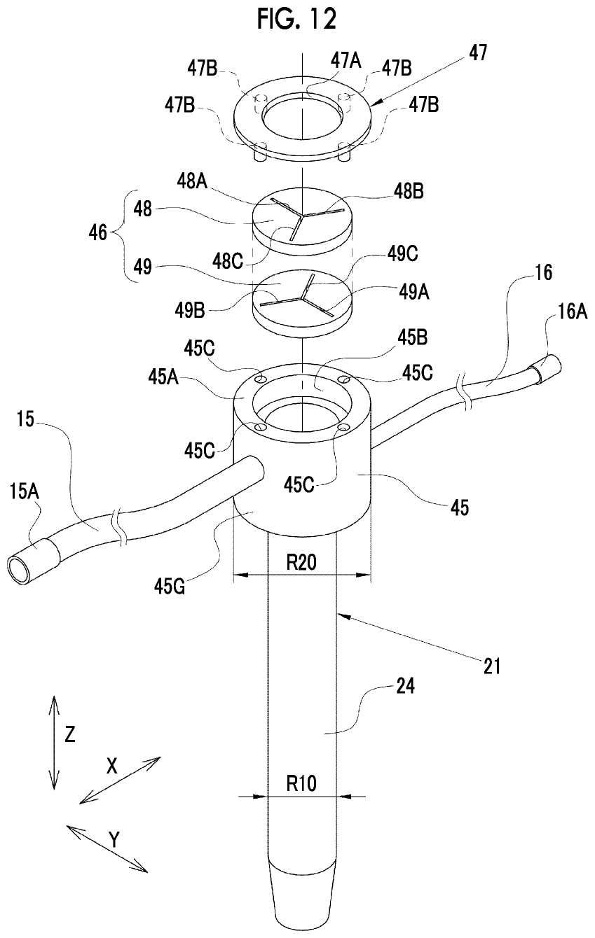

As shown in FIG. 12, the flange portion 45 has a cylindrical shape in which a proximal end 45A is open. The proximal end 45A is formed in a circular shape and has an opening portion 45B formed therein. The opening portion 45B is a circular opening portion formed in conformity with the outer shape of first and second porous members 48 and 49, which will be described below. A fitting hole 45C into which a fitting pin 47B, which will be described below, is fitted is formed around the opening portion 45B.

The opening portion 45B is provided with the fluid suppression member 46. Similarly to the fluid suppression member 22 in the above first embodiment, the fluid suppression member 46 suppresses the passage of the fluid in the pipe line 26 leading to the inside of the body of the subject P in a case where the tubular portion 24 is inserted into the anus. The fluid suppression member 46 includes a first porous member 48 and a second porous member 49.

The first porous member 48 has three first slits 48A to 48C, and the second porous member 49 has three second slits 49A to 49C. The first and second porous members 48 and 49 are formed by forming a flexible porous material into a disk shape. The first slits 48A to 48C and the second slits 49A to 49C are disposed in a Y shape, respectively. It is preferable that the outer diameters of the first and second porous members 48 and 49 have larger dimensions than the outer diameter R10 of the tubular portion 24. In addition, the first and second porous members 48 and 49 are the same as the first and second porous members 31 and 32 in the above first embodiment in terms of materials and functions except that the outer shape is formed in a disk shape, and the description thereof will be omitted.

Additionally, the disposition of the second porous member 49 is the same as that of the first porous member 48 rotated by 180° around the central axis parallel to the insertion direction Z, and the second slits 49A to 49C are disposed at positions rotated by 180° around the central axis parallel to the insertion direction Z with respect to the first slits 48A to 48C. Similarly to the above first embodiment, the cost reduction can be achieved by using two of the same porous members.

An outer diameter R20 of the flange portion 45 is larger than the outer diameter R10 of the tubular portion 24 and protrudes from the outer peripheral surface of the tubular portion 24. Accordingly, in a case where the tubular portion 24 is inserted into the anus, the insertion assist tube 10 is stopped at a position where the flange portion 25 abuts against the body surface of the subject P.

A partition plate and a storage portion (not shown) are formed inside the flange portion 45, similarly to the above first embodiment, but the inside of the flange portion 45 has a circular cross section in conformity with the outer shape of the flange portion 45. The suction tube 15 and the liquid feeding tube 16 are connected to an outer peripheral surface 45G of the flange portion 45. The suction tube 15 and the liquid feeding tube 16 communicate with the storage portion. In addition, the flange portion 45 is the same as the flange portion 25 in the above first embodiment except that the outer shape and the cross-sectional shape are formed in a circular shape, and the description thereof will be omitted. Additionally, the disposition of the suction tube 15 and the liquid feeding tube 16 with respect to the flange portion 25 is the same as that of the above first embodiment.

The retaining member 47 is formed in an annular shape. The retaining member 47 is formed in conformity with the outer shape of the flange portion 45 and is anchored to the proximal end 45A of the flange portion 45. The retaining member 47 has a through-hole 47A and four fitting pins 47B. The through-hole 47A exposes the first slits 48A to 48C of the first porous member 48.

In a case where the fluid suppression member 46 is attached to the insertion assist tube body 21, similarly to the above first embodiment, the opening portion 45B of the flange portion 45 and the outer peripheral surfaces of the first and second porous members 48 and 49 are fitted to each other, and the first and second porous members 48 and 49 are sandwiched between the retaining member 47 and the insertion assist tube body 21. Accordingly, the fluid suppression member 46 is fixed to the insertion assist tube body 21 in a state in which the first and second porous members 48 and 49 are laminated.

As described above, by using the insertion assist tube 10 in which the flange portion 45 is formed in a cylindrical shape, similarly to the above first embodiment, in both a case where the insertion part 3 is inserted through the pipe line 26 and a case where the insertion part 3 is removed from the insertion assist tube 10, the fluid suppression member 46 can suppress the passage of the fluid, and the body fluid and the droplets can be prevented from leaking.

In the fluid suppression members 22 and 46 in the above first and second embodiments, the first porous member 31 or 48 and the second porous member 32 or 49 are each provided with three slits, but the present invention is not limited to this, and four or more slits may be provided. In addition, even in a case where four or more slits are provided, it is preferable that these slits are disposed at equal angle intervals and are connected to each other at the center of the second porous member. In addition, a fluid suppression member made of each porous member provided with one slit in the first modification example may be combined with the flange portion of the second embodiment.

### Third Embodiment

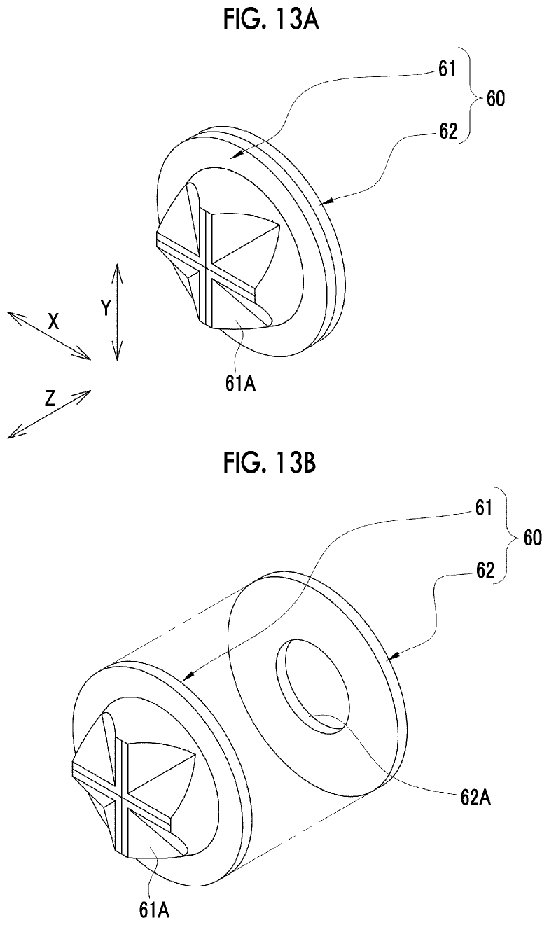

In the above first and second embodiments, an example in which the fluid suppression members 22, 40, and 46 include the first and second porous members in which slits are formed and the like has been shown. However, the present invention is not limited to this as long as the fluid suppression members can suppress the passage of the fluid in both a state in which the insertion part 3 is inserted and a state in which the insertion part 3 is removed. FIGS. 13A and 13B show the configuration of a fluid suppression member 60 having a duckbill valve 61 and a thin plate member 62.

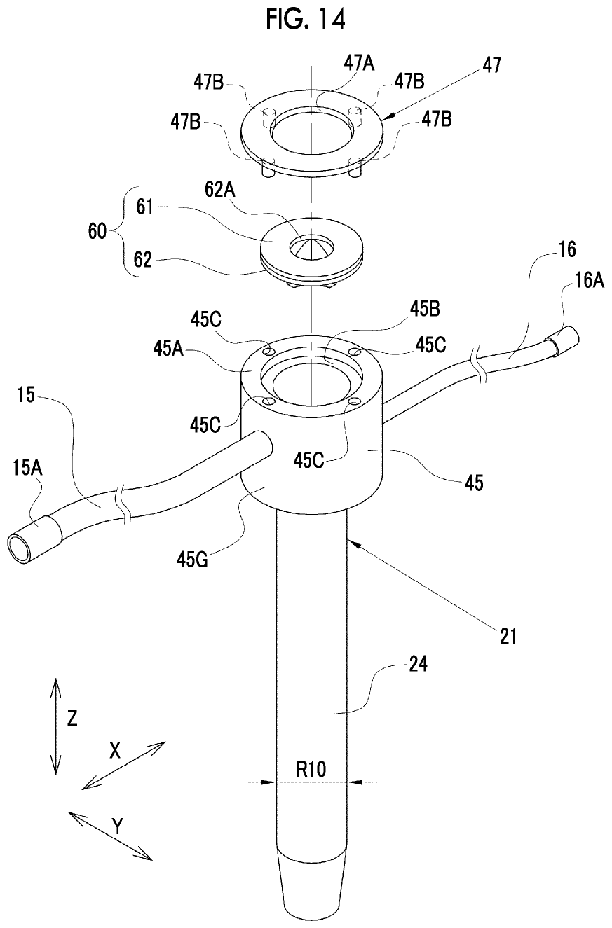

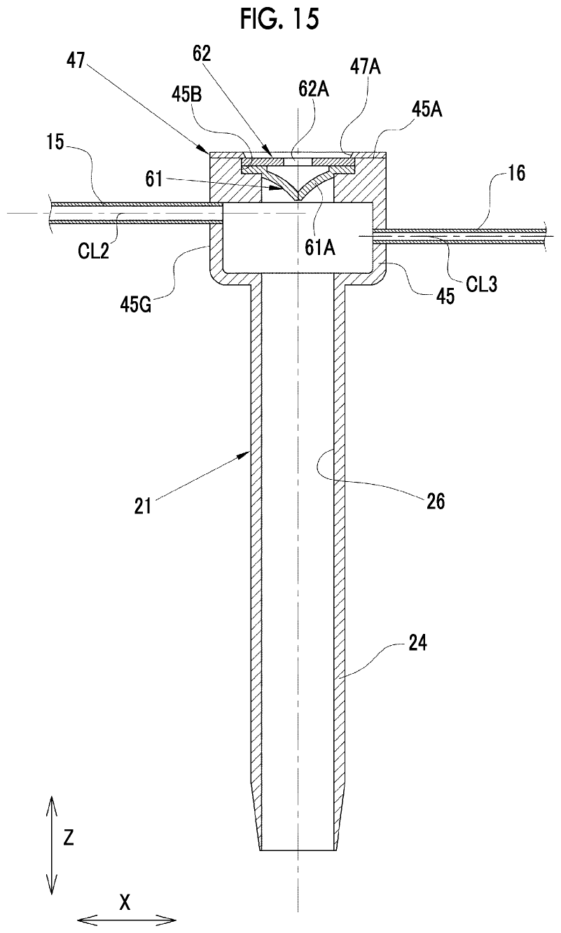

As shown in FIGS. 14 and 15, the fluid suppression member 60 is attached to the opening portion 45B of the insertion assist tube body 21 in a state in which the duckbill valve 61 and the thin plate member 62 are laminated. In addition, in this case, since the duckbill valve 61 and the thin plate member 62 constituting the fluid suppression member 60 have a circular outer shape, it is preferable that the insertion assist tube body 21 provided with the cylindrical flange portion 45 as in the above second embodiment is used. In addition, the insertion assist tube body 21 is the same as the insertion assist tube 10 in the first and second embodiments except that the fluid suppression member 60 is provided, and the description thereof will be omitted. Additionally, the disposition of the suction tube 15 and the liquid feeding tube 16 with respect to the flange portion 45 is the same as those of the above first and second embodiments.

As shown in FIG. 13A, the duckbill valve 61 has a well-known configuration and has a plurality of lip portions 61A that protrude in the insertion direction Z. The duckbill valve 61 is formed of an elastic member such as rubber. Accordingly, the duckbill valve 61 is brought into a state in which the lip portions 61A are in close contact with each other due to an elastic force and are closed, in a state in which the insertion part 3 is not inserted. That is, the passage of the fluid in the fluid suppression member 60 is suppressed. In addition, in a case where the insertion part 3 is inserted through the duckbill valve 61, the lip portions 61A are brought into an open state against the elastic force, and the insertion part 3 is allowed to be inserted.

On the other hand, as shown in FIG. 13B, the thin plate member 62 has a through-hole 62A having an inner diameter that matches the outer diameter of the insertion part 3. The thin plate member 62 is formed of an elastic member such as rubber. Accordingly, in the thin plate member 62, an inner peripheral surface of the through-hole 62A is in close contact with the outer peripheral surface of the insertion part 3 in a state in which the insertion part 3 is inserted. That is, the passage of the fluid in the fluid suppression member 60 is suppressed. It is preferable that the outer diameters of the duckbill valve 61 and the thin plate member 62 have larger dimensions than the outer diameter R10 of the tubular portion 24.

In a case where the fluid suppression member 60 is attached to the insertion assist tube body 21, similarly to the above first embodiment, the opening portion 45B of the flange portion 45 and the outer peripheral surfaces of the duckbill valve 61 and the thin plate member 62 are fitted to each other, and the duckbill valve 61 and the thin plate member 62 are sandwiched between the retaining member 47 and the insertion assist tube body 21. Accordingly, the fluid suppression member 60 is fixed to the insertion assist tube body 21 in a state in which the duckbill valve 61 and the thin plate member 62 are laminated.

By virtue of the above configuration, even in a case where the insertion part 3 is inserted through the fluid suppression member 60 or even in a case where the insertion part 3 is removed, the passage of the fluid can be suppressed, and the body fluid and the droplets can be prevented from leaking from the inside of the insertion assist tube to the outside thereof through the fluid suppression member 60.

### Second Modification Example

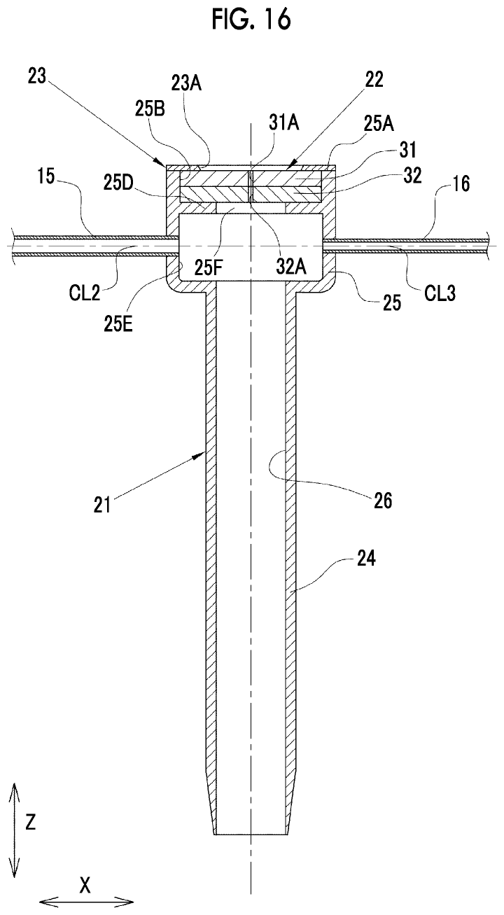

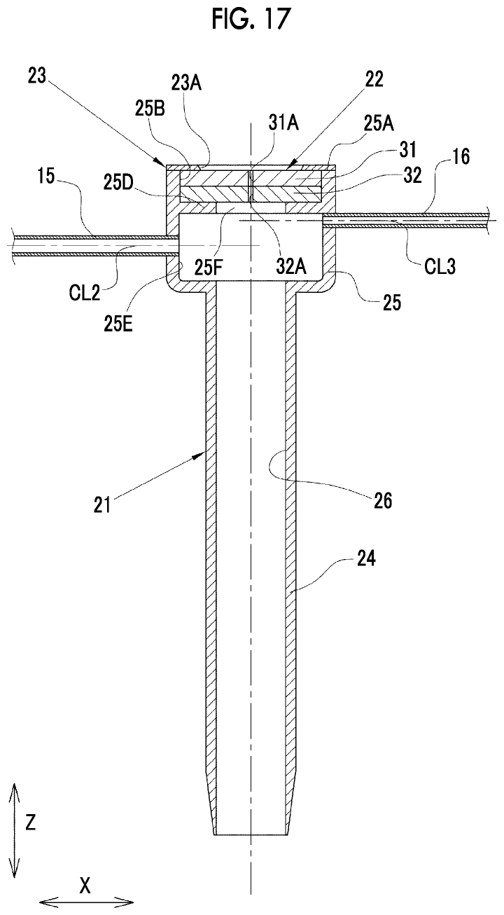

In each of the above-described embodiments, in the suction tube 15 and the liquid feeding tube 16 connected to the flange portions 25 and 45, the center line CL3 of the liquid feeding tube 16 is at the position closer to the inside of the body of the subject P than the center line CL2 of the suction tube 15 is in the insertion direction Z. However, the present invention is not limited thereto. As shown in FIG. 16, the suction tube 15 and the liquid feeding tube 16 may have the same connection position with respect to the flange portion 25 in the insertion direction Z. Alternatively, as shown in FIG. 17, the center line CL2 of the suction tube 15 may be at a position closer to the distal end side than the center line CL3 of the liquid feeding tube 16 is in the insertion direction Z, and closer to the inside of the body of the subject P in a case where the tubular portion 24 is inserted into the anus.

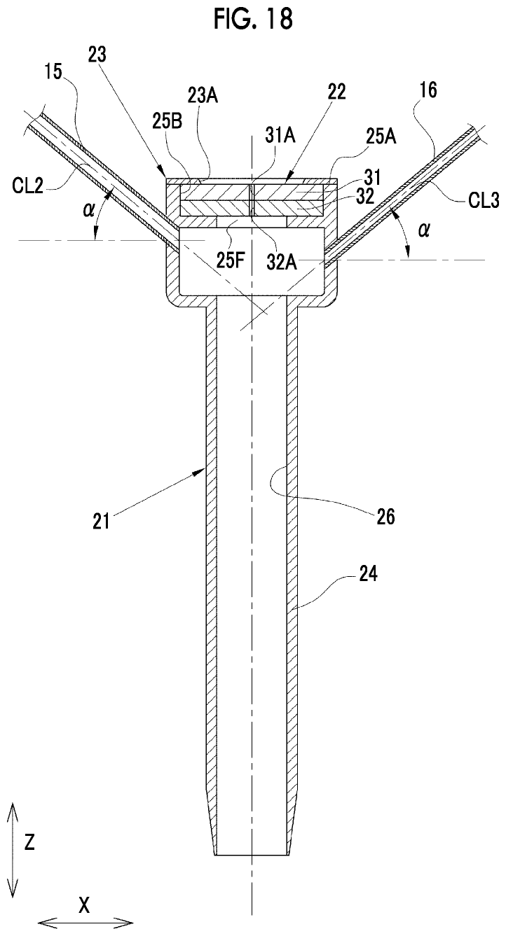

Additionally, in each of the above embodiments, the suction tube 15 and the liquid feeding tube 16 extend in the direction parallel to the left-right direction X with respect to the flange portions 25 and 45. However, the present invention is not limited to this. As shown in FIG. 18, the suction tube 15 and the liquid feeding tube 16 may be inclined with respect to the flange portions 25 and 45 at an inclination angle a toward the proximal end side in the insertion direction Z from a direction perpendicular to the insertion direction Z. Accordingly, in a case where the tubular portion 24 is inserted into the anus, the suction tube 15 and the liquid feeding tube 16 do not come into contact with the subject P.

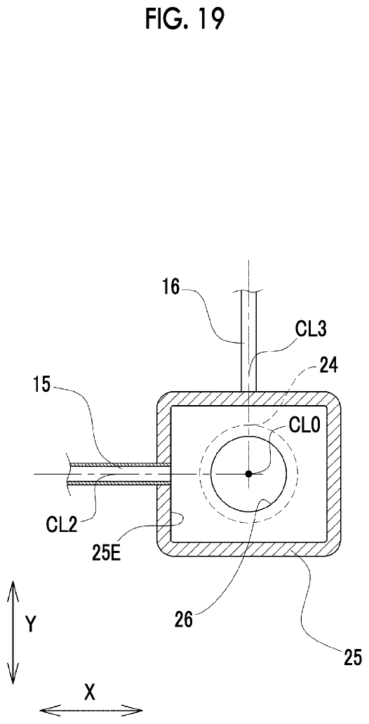

Moreover, in each of the above embodiments, the center line CL3 of the liquid feeding tube 16 is disposed at a position rotated by 180° around the center line CL0 of the tubular portion 24 with respect to the center line CL2 of the suction tube 15. However, the present invention is not limited to this, and the center line CL3 may be disposed at a different position around the center line CL0 of the tubular portion 24. For example, as shown in FIG. 19, the center line CL3 of the liquid feeding tube 16 may be disposed at a position rotated by 90° around the center line CL0 of the tubular portion 24 with respect to the center line CL2 of the suction tube 15.

### EXPLANATION OF REFERENCES

2: endoscope

3: insertion part

4: operating part

5: universal cord

5A: connector

10: insertion assist tube for endoscope

11: processor device

12: light source device

13: display

14: user interface (UI)

15: suction tube

15A: connection connector

16: liquid feeding tube

16A: connection connector

17: suction device

18: liquid feeding device

21: insertion assist tube body

22: fluid suppression member

23: retaining member

23A: through-hole

23B: fitting pin

24: tubular portion

25: flange portion

25A: proximal end

25B: opening portion

25C: fitting hole

25D: partition plate

25E: storage portion

25F: through-hole

25G, 25H: side surface

26: pipe line

31: first porous member

31A to 31C: first slits

31G: gap

32: second porous member

32A to 32C: second slits

32G: gap

40: fluid suppression member

41: first porous member

41A: first slit

42: second porous member

42A: second slit

45: flange portion

45A: proximal end

45B: opening portion

45C: fitting hole

45G: outer peripheral surface

46: fluid suppression member

47: retaining member

47A: through-hole

47B: fitting pin

48: first porous member

48A to 48C: first slits

49: second porous member

49A to 49C: second slits

60: fluid suppression member

61: duckbill valve

61A: lip portion

62: thin plate member

62A: through-hole

AH: anus

CL0, CL1, CL2, CL3: center line

D: doctor

LX0, LX1, LY0, LY1, LZ1: length

P: subject

R10, R20: outer diameter

R11, R12, R13: inner diameter

T: examination table

X: left-right direction

Y: front-back direction

Z: insertion direction

α: inclination angle

What is claimed is:

1. An insertion assist tube for an endoscope, comprising:
an insertion assist tube body that is inserted into a subject in a case where an insertion part of an endoscope is transanally inserted into the subject, the insertion assist tube body having a pipe line through which the insertion part is inserted, and having an opening portion provided at an end of the pipe line on a side located outside a body of the subject in a case where the insertion assist tube body is inserted into the subject; and
a fluid suppression member that is provided in the opening portion to suppress passage of a fluid in the pipe line,
wherein the fluid suppression member has
a first porous member having a first slit parallel to an insertion direction of the insertion part formed therein, and
a second porous member in which a second slit parallel to the insertion direction and intersecting the first slit is formed.

2. The insertion assist tube for an endoscope according to claim 1,
wherein three first slits are formed in the first porous member, and
three second slits are formed in the second porous member.

3. The insertion assist tube for an endoscope according to claim 2,
wherein the first porous member and the second porous member are fixed to the opening portion in a state in which the second slit is disposed at a position rotated by 180° around a central axis parallel to the insertion direction with respect to the first slit.

4. The insertion assist tube for an endoscope according to claim 1,
wherein the first porous member and the second porous member are integrally provided in a state in which the first porous member and the second porous member are laminated.

5. The insertion assist tube for an endoscope according to claim 1, further comprising:
a connection tube that is at a position of the insertion assist tube body closer to the subject than the fluid suppression member is, is disposed at a point located outside the body of the subject, communicates with the pipe line, and is used for connection with an external device.

6. The insertion assist tube for an endoscope according to claim 5,
wherein at least one of the connection tubes is a suction tube that is connected to a suction device serving as the external device and that suctions a liquid in the pipe line.

7. An insertion assist tube for an endoscope, comprising:
an insertion assist tube body that is inserted into a subject in a case where an insertion part of an endoscope is transanally inserted into the subject, the insertion assist tube body having a pipe line through which the insertion part is inserted, and having an opening portion provided at an end of the pipe line on a side located outside a body of the subject in a case where the insertion assist tube body is inserted into the subject;
a fluid suppression member that is provided in the opening portion to suppress passage of a fluid in the pipe line; and
a connection tube that is at a position of the insertion assist tube body closer to the subject than the fluid suppression member is, is disposed at a point located outside the body of the subject, communicates with the pipe line, and is used for connection with an external device,
wherein the fluid suppression member has
a duckbill valve that maintains airtightness of the pipe line in a state in which the insertion part is not inserted, and
a thin plate member that has a through-hole with an inner diameter that matches an outer diameter of the insertion part and maintains the airtightness of the pipe line in a state in which the insertion part is inserted, and
is fixed to the opening portion in a state in which the duckbill valve and the thin plate member are laminated, and
at least one of the connection tubes is a suction tube that is connected to a suction device serving as the external device and that suctions a liquid in the pipe line.

8. The insertion assist tube for an endoscope according to claim 5,
wherein a plurality of the connection tubes are provided.

9. The insertion assist tube for an endoscope according to claim 8,
wherein at least one of the connection tubes is a liquid feeding tube that is connected to a liquid feeding device serving as the external device and that supplies a liquid into the pipe line.

10. The insertion assist tube for an endoscope according to claim 1,
wherein the insertion assist tube body has a tubular portion to be inserted into an anus of the subject, and
the fluid suppression member has a larger dimension than an outer diameter of the tubular portion.

11. The insertion assist tube for an endoscope according to claim 1,
wherein the insertion assist tube body has a tubular portion and a flange portion consecutively installed at the tubular portion, and
the flange portion protrudes from an outer peripheral surface of the tubular portion.
